(19) United States
(12) Patent Application Publication
(10) Pub. No.: US 2018/0126036 A1
(43) Pub. Date: May 10, 2018

(54) DECELLULARIZED BIOMATERIAL FROM MAMMALIAN TISSUE

(71) Applicant: ISE Professional Testing & Consulting Services, Inc., Eustis, FL (US)

(72) Inventor: Ryanne Early, Dallas, TX (US)

(21) Appl. No.: 15/806,122

(22) Filed: Nov. 7, 2017

Related U.S. Application Data
(60) Provisional application No. 62/418,376, filed on Nov. 7, 2016.

(51) Int. Cl. A61L 27/36; A61L 27/58; A61K 35/50

(52) U.S. Cl. CPC A61L 27/3633; A61L 27/3683; A61L 27/58; A61K 35/50; A61L 2300/414; A61L 2300/608; A61L 2300/252; A61L 2300/236; A61L 2430/34; A61L 2430/40; C12N 2533/90; C12N 2533/54; C12N 2533/52; A61L 2300/64

(57) ABSTRACT

The present invention includes a growth factor profile, connective tissue matrix constituents, and immunoprivileged status of equine placental tissue extracellular matrix (ECM) and accompanying cutaneous tissue, plus the presence of antimicrobial peptides there, render equine placental tissue an ideal source for biological scaffolds for xenotransplantation, and optionally adding at least one of: one or more block-copolymers, one or more osteogenic agent or one or more osteoinductive agents.

### CROSS-REFERENCE TO RELATED APPLICATIONS

This application claims priority to U.S. Provisional Application Ser. No. 62/418,376, entitled “Decellularized Biomaterial from Mammalian Tissue,” filed Nov. 7, 2016, the entire contents of which are incorporated herein by reference.

### STATEMENT OF FEDERALLY FUNDED RESEARCH

None.

### TECHNICAL FIELD OF THE INVENTION

The present invention relates in general to the field of decellularized tissue, and more particularly, to compositions and methods for isolating equine decellularized tissue.

### BACKGROUND OF THE INVENTION

Without limiting the scope of the invention, its background is described in connection with tissue decellularization.

Tissue engineering efforts are ongoing to produce methods and materials for replacing biological functions, typically repairing or replacing whole tissues or portions thereof. In this regard, wound treatment and skin repair are areas of predominant focus, as the loss of skin integrity due to illness or injury can lead to chronic, life threatening complications.

Wound healing involves complex interactions between cells, growth factors, and extracellular matrix (ECM) components to reconstitute tissue following injury. The wound healing process in adult mammalian tissue has been well characterized and can be broken down into three stages--inflammation, proliferation, and remodeling.

Typically, in response to an incision or trauma the body conveys blood, blood products, and proteins into the void (also referred to as the cavity or negative space) formed at the wound. During early inflammation, a wound exudate begins to form under the influence of inflammatory mediators and as a result of vasodilation. Fibrin and fibronectin present in clotting blood provide a scaffold over which cells such as keratinocytes, platelets and leukocytes migrate to the wound site. Bacteria and debris are phagocytosed and removed, and growth factors are released that stimulate the migration and division of fibroblasts.

The subsequent stage of wound healing involves new tissue formation as fibrous connective tissue, termed granulation tissue (composed of fibroblasts, macrophages and neovasculature) replaces the fibrin clot. New blood vessels are formed during this stage, and fibroblasts proliferate and produce a provisional ECM by excreting collagen and fibronectin. Nearly all mammalian cells require adhesion to a surface in order to proliferate and function properly. The ECM fulfills this function. Initially, the provisional ECM contains of a network of Type III collagen, a weaker form of collagen that is rapidly produced. This is later replaced by the stronger Type I collagen (which contributes to scar formation). At the same time, re-epithelialization of the epidermis occurs. During this process, epithelial cells proliferate and migrate over the newly forming tissue as proteases such as metalloportineaes (MMPs) and collagenases at the leading edge of the migrating cells help to invade the clot. These enzymes in addition to growth factor signaling (cell-cell interactions) and cell-ECM interactions (signal transduction from interactions between cells, integrins (cell surface receptors), laminin, collagen, fibronectin, and other ECM proteins) stimulate cell migration into the wound and ECM degradation.

Finally, in the remodeling phase, collagen is remodeled and realigned along tension lines and cells that are no longer needed are removed by apoptosis. Wound contraction occurs as fibroblasts transform into myofibroblasts through their interactions with ECM proteins and growth factors. Myofibroblasts then interact with collagen, vitronectin, and other ECM proteins to contract the wound. As the remodeling phase proceeds, fibronectin and hyaluronic acid are replaced by collagen bundles that lend strength to the tissue.

Subjecting the tissue sample to a decellularization process that maintains the structural and functional integrity of the extracellular matrix, by virtue of retaining its fibrous and non-fibrous proteins, glycoaminoglycans (GAGs) and proteoglycans, while removing sufficient cellular components of the sample to reduce or eliminate antigenicity and immunogenicity for xenograft purposes is the manufacturing process (if the tissue is not already acellular from the beginning).

What is needed are new compositions, methods, tissue culture materials, and conditions that promote the growth of skin and other tissue without adverse immunological reactions to the material implanted and that have the strength superior to human or other xenografts.

### SUMMARY OF THE INVENTION

In one embodiment, the present invention includes an acellular or decellularized biomaterial produced by the process that comprises: obtaining placental tissue from an equine animal, which tissue sample comprises extracellular matrix, and decellularizing the sample to retain structural and functional integrity while removing sufficient cellular components of the sample to be suitable for clinical use. In one aspect, the decellularizing comprises subjecting the placental tissue to an alkaline treatment. In another aspect, the process further comprises subjecting the sample to sterilization. In another aspect, the decellularized biomaterial further comprises devitalized cells. In one aspect, the material has a strength greater that the equivalent human tissue.

In another embodiment, the present invention includes a tissue graft comprising extracellular matrix components derived from a placental tissue from an equine animal. In another embodiment, the present invention includes an isolated, decellularized an equine placental tissue extracellular membrane, wherein the membrane is inductive and conductive. In one aspect, the extracellular matrix is alpha-Gal negative. In another aspect, the extracellular matrix includes basement membrane. In another aspect, the extracellular matrix is infused with, coated with, or attached to an agent xenogenic to equine placental tissue that is a growth factor, a cytokine, a chemokine, a protein, a carbohydrate, a sugar, a steroid, an antimicrobial agent, a synthetic polymer, an adhesive, a drug or a human agent. In another aspect, the agent is a cell, optionally a human cell. In another aspect, the extracellular membrane is a sheet. In another aspect, the sheet includes perforations. In another aspect, the extracellular membrane is a dry powder. In another aspect, the extracellular membrane is a reconstituted gel. In another aspect, the extracellular membrane is sterile.

In another embodiment, the present invention includes a package containing an isolated, sterile, decellularized equine placental tissue extracellular membrane, wherein the membrane is inductive and conductive. In one aspect, the isolated, sterile, decellularized equine placental tissue extracellular membrane is a sheet of isolated, decellularized Equine placental tissue equine tissue extracellular membrane. In another aspect, the isolated, sterile, decellularized Equine placental tissue equine tissue extracellular membrane is a dry powder. In another aspect, the isolated, sterile, decellularized equine placental tissue extracellular membrane is a gel.

In another embodiment, the present invention includes a sterile medical implant comprising a sterile, isolated, decellularized equine placental tissue extracellular membrane, wherein the membrane is inductive and conductive. In another aspect, the implant is a biocompatible sheet, mesh, gel, graft, tissue or device.

In another embodiment, the present invention includes a material coated with, impregnated with, encapsulating, or having attached thereto an isolated, sterile, decellularized equine placental tissue extracellular membrane, wherein the membrane is inductive and conductive.

In another embodiment, the present invention includes a tissue culture system comprising: (a) an decellularized equine placental tissue extracellular membrane, (b) tissue culture medium, and (c) mammalian cells, wherein the membrane is inductive and conductive. In another aspect, the mammalian cells are human cells.

In another embodiment, the present invention includes a tissue culture medium conditioned with an isolated isolated, sterile, decellularized equine placental tissue extracellular membrane, wherein the membrane is inductive and conductive.

In another embodiment, the present invention includes a device comprising at least two sheets of an isolated, sterile, decellularized equine placental tissue extracellular membrane laminated to one another, wherein the membrane is inductive and conductive.

In another embodiment, the present invention includes a product prepared by isolating decellularized equine placental tissue extracellular membrane, and sterilizing the decellularized equine placental tissue extracellular membrane, wherein the membrane is inductive and conductive.

In another embodiment, the present invention includes a method of preparing a biologic material comprising: obtaining a tissue sample from an equine, which tissue sample comprises extracellular matrix, and decellularizing the sample forming a decellularized extracellular membrane to remove sufficient cellular components of the sample to reduce or eliminate antigenicity of the biomaterial as a xenograft, wherein the membrane is inductive and conductive. In one aspect, the method further comprises performing the decellularization in a manner to retain structural and functional integrity of the decellularized extracellular matrix membrane sufficient to permit the decellularized extracellular membrane to be useful as a matrix upon and within which cells can grow. In another aspect, the method further comprises homogenizing the decellularized extracellular membrane to form a powder. In another aspect, the method further comprises reconstituting the powder as a gel. In another aspect, the method further comprises sterilizing the decellularized extracellular membrane. In another aspect, the method further comprises attaching the decellularized extracellular membrane to an agent xenogenic to an equine. In another aspect, the extracellular membrane is α-Gal negative. In another aspect, the decellularlized equine extracellular membrane has a surface area of greater than 1,000 cm2. In another aspect, the decellularlized equine extracellular membrane does not collapse during isolation. In another aspect, the decellularlized equine extracellular membrane is not used for ophthalmic use.

In another embodiment, the present invention includes a method of treating a wound, burn, or surgical location with a biologic material comprising: obtaining a decellularized equine placental tissue extracellular membrane to remove sufficient cellular components of the sample to reduce or eliminate antigenicity of the biomaterial as a xenograft, wherein the membrane is inductive and conductive; and placing the decellularized equine placental tissue extracellular membrane in, or, or about the wound, burn, or surgical location to treat the wound, burn, or surgical location. In another aspect, the method further comprises performing the decellularization in a manner to retain structural and functional integrity of the decellularized extracellular matrix membrane sufficient to permit the decellularized extracellular membrane to be useful as a matrix upon and within which cells can grow. In another aspect, the method further comprises homogenizing the decellularized extracellular membrane to form a powder. In another aspect, the method further comprises reconstituting the powder as a gel. In another aspect, the method further comprises sterilizing the decellularized extracellular membrane. In another aspect, the method further comprises attaching the decellularized extracellular membrane to an agent xenogenic to an equine. In another aspect, the decellularlized equine extracellular membrane is not used for ophthalmic use.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show the evaluating of biomarkers for intact basement membrane with collagen IV (FIG. 1A) and laminitis (FIG. 1B) biomarkers that stain positively in processed biomaterial using immunohistochemical antibody staining.

FIG. 2A and FIG. 2B show the evaluation of retained biological properties of underlining extracellular matrix (ECM) with collagen 1 (FIG. 2A) and fibronectin (FIG. 2B) biomarkers when stain positive in processed biomaterial using Immunohistochemical antibody staining.

FIG. 3A and FIG. 3B show the bilateral histoarchitecture is evident as being retained and the absence of cell and cellular debris confirms the process renders the material acellular.

FIG. 3C shows Pico Sirius red birefrigence staining in H shows the relative ratio of collagen 111 to collagen 1 as would be expected in a Neotenic material

FIGS. 4A to 4F are SEM images, (E1, E2, E3), which show the decellurization of equine placental tissue.

FIG. 5 is a graph that shows the degradation of digested collagen normalized to initial weight.

FIG. 6 is a graph that shows axial pull strength for the material of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ideally, transplantable scaffold products should support cell adhesion, proliferation and differentiation and act as an interim synthetic extracellular matrix (ECM) for cells prior to the formation of new tissue. Scaffold materials should be biocompatible, biodegradable and exhibit no, or low, antigenicity. The implant should degrade at a rate roughly equal to that of the new tissue formation. Once implanted, the scaffold must have the mechanical properties necessary to temporarily offer structural support until the new tissue has formed. Additionally, scaffold products must be porous, providing an appropriate path for nutrient transmission and tissue ingrowth. Tissue scaffolds also should promote fast healing and facilitate the development or regeneration of new tissue that resembles normal host tissue in both appearance and function. To this end, implanted scaffold products should offer (i) bioactive stimulation, e.g., protein and molecular signaling, to encourage cell migration, proliferation and differentiation, and (ii) mechanical or structural support for these processes.

The invention is useful for preparation of a variety of human and animal personal care (cosmeceuticals) and healthcare products (medical devices such as implants, diagnostic tools, pharmaceutical preparations, medical research product, etc.). In certain embodiments, the composition taught herein can also include using the biomaterial combined with a block copolymer (e.g., poloxamer). In another embodiment, the novel substrates may using the methods of the present invention may also include one or more osteogenic and/or osteoinductive agents. In other embodiment, the composition may include both the block co-polymers and the one or more osteogenic and/or osteoinductive agents.

The present invention find particular advantages over known extracellular matrices, such as those obtained from cadaveric human tissue, in that the present invention provides a material with a very large surface area (e.g., greater than 50, 75, 100, 125, 150, 200, 250, 300, 400, 500, 600, 700, 800, 900, 1,000, 2,000, 3000, or 4,000 cm²) and a mechanical strength that permits use of the material for, e.g., use in mechanically challenging surgeries such as hernias, tendon, or other orthopedic surgical needs.

Further, the present invention finds particular uses because it has been found, surprisingly, that the equine source material provides a rare combination of being both inductive and conductive.

As used herein, the term “inductive” refers to a material that is induces to the growth of certain types of cells into the material, e.g., stem cells from skin, tendon, bone, or other materials. For example, the term “osteoinductive” refers to a material that when inserted into a bone or adjacent a bone would induce the growth of osteoclasts and other such cells into this osteoinductive material.

As used therein, the term “conductive” refers to a material that provides a scaffold that provides mechanical strength at the location of insertion. When a material is “osteoconductive” this refers to a material that provide mechanical support in an area in or adjacent to a bone that provides a material that provides a scaffold for bone growth.

As used therein, the term “protective” refers to a material that provides a scaffold that provides mechanical strength at the location of insertion but also provides bioprotection against the environment.

The equine material of the present invention has been found to be both inductive and conductive when used as a biomaterial. Further, because of its large surface area and strength, it was also found to be protective when used as a replacement for, e.g., skin, or to enhance the strength of damaged skin and help protect the underlying tissue from infection and other debris. The present invention finds particular uses in burn victims and victims of accidents where a significant area of skin has been removed, for example, an area greater than 50 cm², or even greater than 1,000 cm². The present invention finds particular uses as a skin scaffold, for use after surgeries, e.g., hernia, orthopedic, or other surgeries that require a material with a strength that exceeds that of material from human donors. Other uses include, e.g., wound healing, tissue closure, bulking tissue, preventing tissue adhesion, providing structural support to tissue, providing a protective barrier, and/or correcting a defect.

As used herein, the terms “decellularization” or “acellular” refers to biomaterial produced by decellularizing a tissue sample obtained from equine placental tissue. The primary constituent of the resulting equine placental biomaterial is an extracellular membrane (ECM), possibly with devitalized epithelial cells, which can retain moisture and otherwise protect a wound-healing environment.

Equine placental tissue is used as a starting material for the present invention. Thus, the starting material that is subjected to decellularization can comprise equine placental tissue dermis and basement membrane, with or without epidermis. Even upon decellularization, moreover, the biomaterial of the invention can comprise, with the ECM, adjacent epithelial cells that may be rendered non-viable by the process. Alternatively, non-cutaneous equine placental tissues can serve as the starting material of the invention, particularly those comprising a basement membrane or epithelial tissues. Tissues that contain substantial amounts of fibrous connective tissue, such as cartilage, tendon, bone, dura mater and fascia, also are illustrative of appropriate starting materials of the present invention.

In one example, conventional decellularization methodology can be used on the equine placental tissue to remove immunogenic cellular antigens that can induce an inflammatory response or immune-mediated tissue rejection, while preserving the structural integrity and composition of the associated ECM. Generally, ECM structural components, many if not all of which remain intact following decellularization, are well-tolerated by xenogeneic recipients. ECM components that may be present in the final biomaterial of the invention include proteins such as collagen (e.g., fibrous collagen I and collagen III, as well non-fibrous collagen IV, collagen V and collagen VII), elastin, fibronectin, laminin, vitronectin, thrombosponsdins, osteopontin and tenascins, plus GAGs (e.g., the proteoglycans, decoran and versican and sulfated GAGs, e.g., heparin sulfate, keratan sulfate, dermatan sulfate and chondroitin sulfate) and growth factors such VEGF, BMP, TGF and FGF. For some indications the post-decellularization material comprises at least collagen IV, laminin, sulfated GAGs and one or more growth factors in amounts that approximate pre-decellularization levels when viewed via histological and immunohistological staining.

Suitable techniques for decellularizing tissues, pursuant to the invention, include physical methods such as freezing, direct pressure application, sonication, and agitation. In addition or in the alternative, chemical methods can be employed, such as alkaline and acid treatments, application of detergents (including amphoteric, cationic, anionic and non-ionic detergents), organic solvents, hypotonic or hypertonic solutions and chelating agents. Enzymatic approaches including protease digestion and treatment with one or more nucleases also may be used to decellularize equine placental tissue. In addition or alternatively, the equine placental tissue is subjected to cleaning, sterilization, disinfection, antibiotic treatment and/or viral inactivation.

According to one aspect of the invention, a biomaterial is provided. The material is produced by the process that includes: (a) obtaining an equine placenta tissue sample from one of the equine placental tissues, which tissue sample comprises extracellular matrix, and (b) decellularizing the sample to retain structural and functional integrity while removing sufficient cellular components of the sample to reduce or eliminate antigenicity of the biomaterial as a xenograft. In some embodiments, decellularizing comprises subjecting the tissue sample to an alkaline treatment. In embodiments, the process can further comprise subjecting said sample to sterilization. In embodiments, the process can further comprise devitalizing cells.

According to one aspect of the invention, a tissue graft is provided. The graft includes extracellular matrix components derived from equine placental tissue. In embodiments, the extracellular matrix components are substantially free of components that induce an immune response when implanted as a xenograft. In embodiments, the extracellular matrix components are non-toxic.

### Definitions

As used herein, the term “equine placental tissue” refers to maternal and fetal equine birth tissues expelled during the birth process to include, but not limited to, all tissues related to the birth of an equine (such as placenta body, umbilical cord, amnion, chorioallantois, amnion, allantoamnion, extra-amnionic cord, urachus, yolk, decidua, and all related vessels, membranes (from the underlying matrix)), fluids and tissues.

As used herein, the term “extracellular matrix (ECM)” refers to maternal and fetal equine birth tissues that include the basement membrane.

As used herein, the term “biocompatible” refers to a composition and its normal degradation products in vivo are substantially non-toxic and non-carcinogenic in a subject within useful, practical and/or acceptable tolerances.

As used herein, the term “bytocompatible” refers to a composition can sustain the viability and growth of a population of cells.

As used herein, the term “decellularized ECM” or “acellular ECM” refers to an extra cellular matrix sufficiently free of cellular components to eliminate or reduce antigenicity of the extra cellular matrix to an extent where the matrix would be considered non-toxic as a xenograft.

As used herein, the term “isolated” when used in connection with the ECM of the invention refers to tissue separated from other Equine placental tissue.

As used herein, the term “non-toxic” refers to a composition, when implanted in a subject, causes little or no adverse reaction or substantial harm to cells and tissues in the body, and does not cause a substantial adverse reaction or substantial harm to cells and tissues in the body, for instance, the composition does not cause necrosis, an infection, or a substantial immune response resulting in harm to tissues from the implanted or applied composition.

As used herein, the term “progenitor cell” refers to a cell that can differentiate under certain conditions into a more-differentiated cell type. Non-limiting examples of progenitor cells include stem cells that may be totipotent, pluripotent, multipotent stem cells, or referred to as progenitor cells. Additional non-limiting examples of progenitor cells include perivascular stem cells, blastema cells, and multilineage progenitor cells.

As used herein, the term “retain structural and functional integrity” used in connection with the ECM of the invention refers to retaining sufficient structure and function to permit and support the use of the matrix as a substrate for the growth of cells in vivo or in vitro.

As used herein, the term “subject” refers to an animal. In some embodiments the animal is a mammal. The mammal can be a dog, cat, a horse, a cow, a goat, a sheep, a pig or a non-human primate. In any embodiment the mammal can be a human.

As used herein, the term “treatment” or “treating” refers to the administration or application to a subject by any suitable placement, insertion, layering, stitching, or other medical regimen and route of administration of the composition with the object of achieving a desirable clinical/medical end-point, such as assisting in wound healing, tissue closure, bulking tissue, preventing tissue adhesion, providing structural support to tissue, providing a protective barrier, correcting a defect, etc.

As used herein, the term “equine placental tissue fraction derived from decellularized Equine placental tissues ECM” refers to an extract or isolate of decellularized equine placental tissue ECM maintaining sufficient characteristics of an Equine placental tissue in terms of chemical structure and/or relative chemical concentrations of two (or three, or four, or five or more) chemical entities in the extract or isolate to distinguish the extract as obtained from an Equine placental tissue by any one or more of electron microscopy, HPLC, immunohistochemistry, and the like.

General Preparative Methodology. According to the invention, equine placental tissue samples obtained for decellularization can be treated in the manner detailed in US2008/0046095 or US2010/0104539, relevant portions incorporated herein by reference. Thus, tissue samples may be subjected to cleaning and chemical decontamination. Briefly, a tissue sample is washed for approximately 10 to 30 minutes in a sterile basin containing 18% NaCl (hyperisotonic saline) solution that is at or near room temperature. Visible cellular debris, such as epithelial cells adjacent to the tissue basement membrane, is gently scrubbed away using a sterile sponge to expose the basement membrane. Using a blunt instrument, a cell scraper or sterile gauze, any residual debris or contamination also is removed. Other techniques including, but not limited to, freezing the membrane, physical removal using a cell scraper, or exposing the cells to nonionic detergents, anionic detergents, and nucleases also may be used to remove cells. In one embodiment, equine placental tissue is decellularized using alkaline treatment.

The tissue is placed into a sterile container, such as a Nalgene jar, for the next step of chemical decontamination. Thus, each container is aseptically filled with 18% saline solution and sealed (or closed with a top). The containers then are placed on a rocker platform and agitated for between 30 and 90 minutes, which further cleans the tissue of contaminants.

In a sterile environment using sterile forceps, the tissue is gently removed from the container containing the 18% hyperisotonic saline solution and placed into an empty container. This empty container with the tissue is then aseptically filled with a pre-mixed antibiotic solution. Preferably, the premixed antibiotic solution is comprised of a cocktail of antibiotics, such as Streptomycin Sulfate and Gentamicin Sulfate. Other antibiotics, such as Polymyxin B Sulfate and Bacitracin, or similar antibiotics available now or in the future, are suitable as well. It is preferred that the antibiotic solution be at room temperature when added so that it does not change the temperature of or otherwise damage the tissue. This container containing the tissue and antibiotics is then sealed or closed and placed on a rocker platform and agitated for, preferably, between 60 and 90 minutes. Such rocking or agitation of the tissue within the antibiotic solution further cleans the tissue of contaminants and bacteria.

In a sterile environment, the container is opened and, using sterile forceps, the tissue is gently removed and placed in a sterile basin containing sterile water or normal saline (0.9% saline solution). The tissue is allowed to soak in place in the sterile water/normal saline solution for at least 10 to 15 minutes. The tissue may be slightly agitated to facilitate removal of the antibiotic solution and any other contaminants from the tissue.

In some cases, the present invention involves treating equine placental tissue using a chemical sterilization methodology, as illustrated the TUTAPLAST® and ALLOWASH® procedures, optionally in combination with mechanical processes that gently agitate chemical agents, as in the BIOCLEANSE® system. Thus, equine placental tissue is subjected to oxidative and/or alkaline treatments as well as osmotic treatment to break down cell walls, to inactivate pathogens, and to remove bacteria. In addition, tissue may be subjected to delipidization, solvent dehydration (to permit room temperature storage of tissue without damaging the collagen structure) and/or low-dose gamma irradiation to ensure sterility of the final product.

Efficient cell removal upon decellularization can be verified by various known methods, including histological analyses to detect nuclear and cytoplasmic structures, immunohistochemical or immunofluorescent assaying for indicative intracellular proteins, and DNA detection. The nature of desirable components in the final Equine placental tissue-derived scaffold biomaterial varies depending on the clinical indication being treated. Once a particular indication is identified, the knowledgeable clinician can determine which components in the equine placental tissue sample should be retained in the final scaffold product, and standard methodology can be employed to ensure that the desired components are present following decellularization.

Samples may be viewed histologically before, during, and/or after decellularization to monitor the process and to confirm that the desired degree of cellular component removal is reached. For instance, tissues can be analyzed for cytoskeletal content to gauge sufficient decellularization. Intracellular protein content also may be assayed to determine if decellularization is sufficient. In addition, the tissue sample thickness and chemical makeup may be monitored to determine when sufficient decellularization has been achieved. Periodic monitoring during processing allows for a real time response to the observed tissue properties.

In some cases, a sufficiently decellularized tissue comprises no more than 50 ng dsDNA per mg ECM dry weight. Alternatively, for some indications, a sufficiently decellularized tissue lacks visible nuclear material in a tissue section stained with 4′,6-diamindino-2-phenylindole (DAPI) or haematoxyilin and eosin (H&E).

In scenarios where removal of an adjacent epithelial cell layer is required, the presence or absence of epithelial cells remaining in the sample can be evaluated using techniques known in the art. For example, after removal of the epithelial cell layer, a representative tissue sample from the processing lot is placed onto a standard microscope examination slide. The tissue sample is then stained using Eosin Y Stain and evaluated as described below. The sample is then covered and allowed to stand. Once an adequate amount of time has passed to allow for staining, visual observation is done under magnification. The presence of cells and cellular material will appear darker than the areas which have been de-epithelialized.

Once cellular removal has progressed sufficiently, conventional methods are employed to confirm the retention of desired structural and functional properties of the remaining ECM scaffold. The specific structural testing that should be conducted depends on the intended clinical application of the final scaffold product. In some cases, the equine placental tissue starting material may be monitored before, during, and after decellularization to ensure that the desired structural components and configuration are maintained in the final product.

One method for determining whether the desired ECM components are present involves staining parallel tissue sections and examining them histologically to determine whether the desired constituents and structural orientation of the equine placental tissue have been preserved. For instance, equine placental tissue can be stained with H&E and immunoperoxidase stain for laminin to assess preservation of ECM and laminin. In general, the three-dimensional configuration of ECM components remaining in the final biomaterial scaffold product should approximate that of pre-decellularized material when viewed via histological staining. Another component one can assay for is AMPS, as the ECM of the invention is rich in AMPS.

Accordingly, the Equine placental tissue-derived biomaterial of the invention comprises ECM components useful for directing enhanced re-epithelialization and promoting efficient tissue regeneration or wound healing. The inventive biomaterial also serves as a matrix and reservoir for bioactive peptides such as growth factors, adhesion proteins and AMPS. Accordingly, the biomaterial functions effectively as a biological scaffold for tissue regeneration, providing both the necessary bioactive stimulation and structural support. The product can be used as is, cut into smaller pieces or shapes, laminated to itself or other materials, pre-punctured to provide openings for securing attachments, formed into desired three dimensional shapes, as well as other formats, discussed in more detail below.

Powders and Gels. In embodiments, the scaffold can be further processed into small grains or a powder. The fine particles can be hydrated in water, saline or a suitable buffer or medium to produce a paste or gel. This fine material, paste or gel produced from it may be used for a multitude of purposes, described in greater detail below.

Although numerous methods exist, two exemplary methods may be used to produce a particulate form of the scaffold. The first method involved lyophilizing the disinfected material and then immersing the sample in liquid nitrogen. The snap frozen material is then reduced to small pieces with a blender so that the particles are small enough to be placed in a rotary knife mill, such as a Wiley mill. A #60 screen can be used to restrict the collected powder size to a desired size, for example less than 250 mm. A Sonic sifter or other classification device can be used to remove larger particles and/or to obtain a particle size distribution within a desired range. A second method is similar to the previous method except the sample is first soaked in a 30% (w/v) NaCl solution for 5 min. The material is then snap frozen in liquid nitrogen to precipitate salt crystals, and lyophilized to remove residual water. This material is then comminuted as described in above. By precipitating NaCl within the sample, it is expected that the embedded salt crystals would cause the material to fracture into more uniformly sized particles. The particles are then suspended in deionized water and centrifuged for 5 min at 1,000 rpm three times to remove the NaCl. The suspension is snap frozen and lyophilized again. Finally, the powder is placed in a rotary knife mill to disaggregate the individual particles. The powder can be hydrated to create a gel, with or without other gelling materials to supplement gelling.

The powder, paste or gel can be applied without further processing to treat a subject. It can be sprayed, painted, injected or otherwise applied to a wound or surgical site. The gel can be shaped. The powder, paste or gel also can be placed inside a “bag”, such as a polymeric synthetic material or a ECM sheet as described herein to produce a larger three-dimensional structure, such as an orthopedic implant for cartilage repair (e.g., knee or TMJ cartilage repair) or an implant for breast reconstruction or augmentation. In such a case, a bag of a desirable size and shape is formed from sheets of ECM material or other biocompatible polymeric material, and then the bag or cover can be filled with the tissue-derived powder or gel described herein. The shape of the device or implant can vary with its intended use. The bag may be molded into a useful shape by any useful molding technique, such as the shape of cartilage for the ear, nose, knee, TMJ, rib, etc., prior to filling the molded bag with the scaffold material described herein. In one example, a biodegradable polymeric matrix (e.g., PEUU or PEEUU) is sprayed or electrodeposited onto a mold. The resultant molded cover can then be filled with the material. Heat, for example, may be used to seal the cover.

Additives. Generally, the agents include any agent useful in cell culture or as a therapeutic or therapeutic adjuvant. The agents can be coated on, infused into or otherwise covalently or non-covalently attached to or incorporated onto or into the ECM of the invention. The agents also can be otherwise combined with a product that contains the ECM, for example, as by mixing powders of the agent and ECM together. Each agent may be used alone with the ECM of the invention or in combination with other agents. Non-limiting examples of such agents include antimicrobial agents, growth factors, cytokines, chemokines, emollients, retinoids, steroids, and cells, including but not limited to the subject's own cells.

In certain non-limiting embodiments, the agent is a growth factor. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factorsα and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

In certain non-limiting embodiments, the therapeutic agent is an antimicrobial agent, such as, without limitation, an anti-microbial peptide, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

In certain non-limiting embodiments, the therapeutic agent is an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin.

Other drugs that may promote wound healing and/or tissue regeneration may also be included. The agent may be dispersed within the scaffold by any useful method, e.g., by adsorption and/or absorption. For example, the therapeutic agent may be dissolved in a solvent (e.g., DMSO) and added to the scaffolding. In another embodiment, the agent is mixed with a carrier polymer (e.g., polylactic-glycolic acid microparticles, agarose, a poly(ester urethane) urea elastomer (PEUU) or a poly(ether ester urethane) urea elastomer (PEEUU)), which is subsequently dispersed within or applied to the scaffold. By blending the agent with a carrier polymer or elastomeric polymer, the rate of release of the therapeutic agent may be controlled by the rate of polymer degradation and/or by release from the polymer by diffusion or otherwise. Likewise, a therapeutic agent may be provided in any dissolvable matrix for extended release, as are known in the pharmaceutical arts, including sugar or polysaccharide matrices. The agent also may be included with the powdered ECM and gelled with the powdered ECM. The agent may be covalently attached to the ECM of the invention. The foregoing are meant to be non-limiting examples.

Extracts. In addition to the decellularized ECM in its native state or ground as a particulate or powder, the invention also provides extracts and isolates of the same. As mentioned above, the Equine placental tissue ECM is loaded with antimicrobial peptides, growth promoting factors, collagen and laminins, and Equine placental tissue fractions of the ECM are useful according to the invention.

Extraction buffers are well known in the art. One such buffer is 4 M guanidine and 2 M urea each prepared in 50 mM Tris-HCl, pH 7.4. The powder form of the ECM can be suspended in the relevant extraction buffer (e.g., 25% w/v) containing phenylmethyl sulphonyl fluoride, N-ethylmaleimide, and benzamidine (protease inhibitors) each at 1 mM and vigorously stirred for 24 hours at 4° C. The extraction mixture can then be centrifuged and the supernatant collected. The insoluble material can be washed in the extraction buffer, centrifuged, and the wash combined with the original supernatant. The supernatant can be dialyzed against deionized water. The dialysate can then be centrifuged to remove any insoluble material and the supernatant used immediately or lyophilized for long term storage. Such an isolate will contain growth factors in concentrations specific to Equine placental tissues.

In another aspect, the extraction is done by conditioning medium. A method of making Equine placental tissue-specific extract by taking the powdered ECM, forming a solution thereby generating a supernatant and a particulate, wherein the supernatant is an extract and isolating the extract from the particulate. One also could grow cells on the ECM, and isolate the supernatant after a period of time of cell growth.

Synthetic Materials. Synthetic biocompatible and cyto-compatable material can be combined with the ECM, such as, for example, (a) a structural support for a sheet or a gel of the ECM, (b) a structural support for shaping the ECM, (c) a coating for the ECM (or a coating containing the particulate ECM), a supplemental gelling agent, or (d) a sustained release material for the particulate ECM or an isolate thereof. Such polymers have been known to be applied to other ECM materials as a backing sheet, including materials that are themselves biodegradable. Suitable synthetic material for a matrix can be biocompatible to preclude migration and immunological complications, and can be able to support cell growth and differentiated cell function. Some are resorbable, allowing for a completely natural tissue replacement. Some can be configurable into a variety of shapes and have sufficient strength to prevent collapse upon implantation. Studies indicate that the biodegradable polyester polymers made of polyglycolic acid fulfill all of these criteria (Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991)). Other synthetic biodegradable support matrices include synthetic polymers such as polyanhydrides, polyorthoesters, and polylactic acid. Further examples of synthetic polymers and methods of incorporating or embedding cells into these matrices are also known in the art. See e.g., U.S. Pat. Nos. 4,298,002 and 5,308,7.

As a non-limiting example, the powder may be formulated with tri-block co-polymers. See international published application WO2012131104 and WO2012131106, each of which is incorporated herein by reference in its entirety. Other examples include poloxamers, which are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade name Pluronics (BASF). Certain poloxamers are useful as sustained release materials for pharmaceuticals.

Particles of the invention also may be encapsulated into a polymer, hydrogel and/or surgical sealant. As a non-limiting example, the polymer, hydrogel or surgical sealant may be PLGA, ethylene vinyl acetate (EVAc), POLOXAMER®, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.). In another embodiment, the particle may be encapsulated into any polymer known in the art which may form a gel when injected into a subject. As another non-limiting example, the particle may be encapsulated into a polymer matrix which may be biodegradable. Additional examples of polymers for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®.

Uses. The decellularized Equine placental tissue ECMs described herein are useful for growing cells, tissues, organs in virtually any in vivo, ex vivo, or in vitro use. The ECMs can be used as a substrate to facilitate the growth and/or differentiation of cells. In vitro, the ECMs are useful as a cell growth substrate to support the growth in culture of cells, including virtually any type of cells or cell-lines, including stem cells, progenitor cells or differentiated cells. In one embodiment, the cells are cancer cells. In one embodiment, the cancer cells form nodules when grown on the ECMs. Cells on the substrate also may be grown into tissue, organ or body part precursors, or even mature tissues or structures. Cells grown on ECMs may be used for implantation, for wound dressings, for in vitro drug testing, for modeling differentiation, etc. The cells may be matched in tissue cell type to the ECM or unmatched. The cells are xenogenic.

The Equine placental tissue ECM of the invention is useful in vivo as a cell growth scaffold for tissue growth for any useful purpose, including repair, replacement or augmentation of tissue in a subject in either humans or animals. For example, the materials are useful in repair and/or replacement of tissue lost or damaged during trauma or surgery, for example in loss of tissue after tumor removal. The materials are useful for structural repair, such as inguinal hernia repair, parastomal reinforcement, soft tissue reinforcement, surgical staple-line reinforcement during, for example, bariatric surgery or lung resection, umbilical hernia grafts, Peyronie's repair grafts, incision grafts and fistula plugs. The materials are useful for wound dressings, such as for burns, graft and split-thickness graft coverings, ulcers including decubitis ulcers and dermal abrasion procedures. The materials are useful for cosmetic purposes, for example in breast, lip or buttock augmentation. An aspect of the invention particularly appealing for anti-adhesion surgical uses is the properties of the basement membrane, which inhibit or prevent adhesion. The presence of the AMPs make the ECM of the invention particularly well suited for the foregoing applications.

As mentioned above, the materials described herein can be molded or contained within a structure to form desired shapes, such as, for cartilage repair or replacement by seeding the material with, e.g., chondrocytes and/or chondroprogenitor cells. The materials can be ground into a powder and used to reconstitute and/or form gels, as cell culture additives, as a powder, spray, liquid, suspension or coating for application to (a) a wound, (b) an implant, (c) a wound dressing, etc.

In one embodiment, for example, adipose stem cells are propagated in the cell growth scaffolds described herein. Adipose stem cells are of mesodermal origin. They typically are pluripotent, and have the capacity to develop into mesodermal tissues, such as: mature adipose tissue; bone; heart, including, without limitation, pericardium, epicardium, epimyocardium, myocardium, pericardium, and valve tissue; dermal connective tissue; hemangial tissues; muscle tissues; urogenital tissues; pleural and peritoneal tissues; viscera; mesodermal glandular tissues; and stromal tissues. The cells not only can differentiate into mature (fully differentiated) cells, they also can differentiate into an appropriate precursor cell (for example and without limitation, preadipocytes, premyocytes, preosteocytes). Also, depending on the culture conditions, the cells can also exhibit developmental phenotypes such as embryonic, fetal, hematopoetic, neurogenic, or neuralgiagenic developmental phenotypes.

In one embodiment, a subject's own cells are dispersed within the matrix. For example, in the production of cartilaginous tissue, chondrocytes and/or chondroprogenitor cells can be dispersed within the matrix and optionally grown ex vivo prior to implantation. Likewise, skin cells of a subject can be dispersed within the scaffolding prior to implantation on a damaged skin surface of a subject, such as a burn or abrasion.

When used as a gel, a non-limiting example is injecting the gel into a subject at a desirable site, such as in a wound. In one instance, the gel can be injected in a bone breakage or in a hole drilled in bone to facilitate repair and/or adhesion of structures, such as replacement ligaments, to the bone. In another use, finely comminuted particles can be sprayed onto a surface of a subject, such as in the case of large surface abrasions or burns. The scaffold can also be sprayed onto skin sutures to inhibit scarring. The equine placental decellularized ECM of the invention can be place or sutured in place inside the body at a surgical site such as mentioned above. All of these treatments are embraced by the present invention.

Equine placental tissue decellularized ECM can be used also for sustained delivery of therapeutic molecules, proteins or metabolites, to a site in a host. See, for example, U.S. 2004/0181240, which describes an amniotic membrane covering for a tissue surface that may prevent adhesions, exclude bacteria or inhibit bacterial activity, or to promote healing or growth of tissue, and U.S. Pat. No. 4,361,552, relevant portions incorporated herein by reference, which pertains to the preparation of cross-linked amnion membranes and their use in methods for treating burns and wounds. The ECMs of the invention can be used in the same manner.

Pharmaceutical Formulations. Although the descriptions of pharmaceutical compositions provided herein are principally directed to pharmaceutical compositions that are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

The pharmaceutical compositions described herein may be prepared by any method known in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-use configuration.

The ECM in accordance with the invention may be prepared, packaged, and/or sold in bulk, as a single unit dose, and/or as a plurality of single unit doses. For example, the composition may comprise between 0.1% and 100% (w/w) of the ECM. When other active agents are included, relative amounts of agents combined with the ECM of the invention will be known to those of ordinary skill in the art, similar to those amounts used in combination with ECM as formulated in the prior art. Relative amounts also may vary, depending upon the identity, size, and/or condition of the subject being treated and further depending upon the route by which the ECM is to be administered.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art. See Remington: THE SCIENCE AND PRACTICE OF PHARMACY (21st Ed.), A. R. Gennaro, Lippincott, Williams & Wilkins (Baltimore, Md., 2006); incorporated herein by reference in its entirety.

Equine placental tissue samples were obtained for decellularization and treated in the manner detailed in US 2008/0046095 or US 2010/0104539, relevant portions incorporated herein by reference. Tissue samples were subjected to cleaning and chemical decontamination. Finally, a tissue sample was washed for approximately 10 to 30 minutes in a sterile basin containing 18% NaCl (hyperisotonic saline) solution that is at or near room temperature. Visible cellular debris, such as epithelial cells adjacent to the tissue basement membrane, is gently scrubbed away using a sterile sponge to expose the basement membrane. Using a blunt instrument, a cell scraper or sterile gauze, any residual debris or contamination was also removed, and the tissue is washed. The tissue was ready to sterilize.

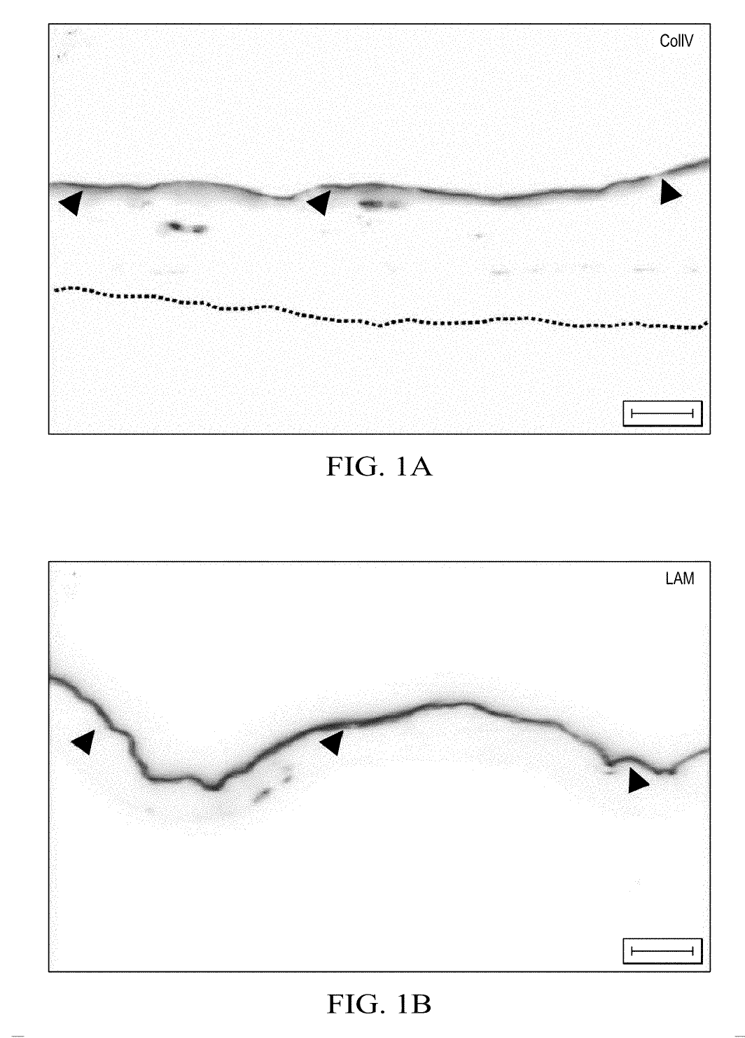

FIGS. 1A and 1B show the evaluating of biomarkers for intact basement membrane with collagen IV (FIG. 1A) and laminitis (FIG. 1B) biomarkers that stain positively in processed biomaterial using immunohistochemical antibody staining.

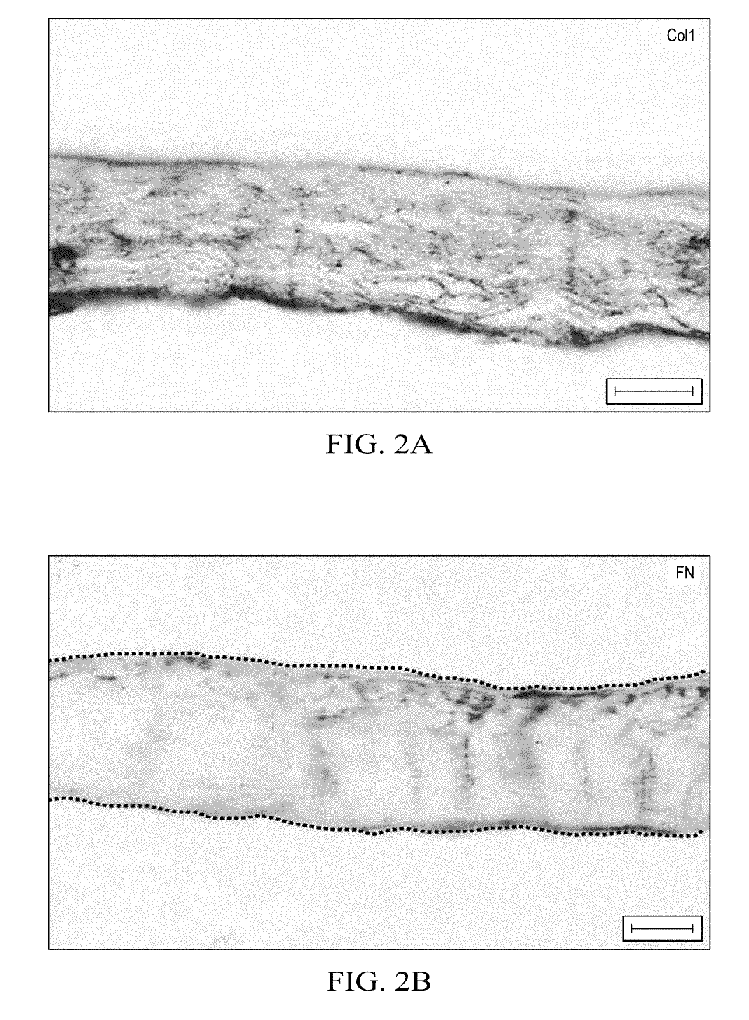

FIG. 2A and FIG. 2B show the evaluation of retained biological properties of underlining extracellular matrix (ECM) with collagen 1 (FIG. 2A) and fibronectin (FIG. 2B) biomarkers when stain positive in processed biomaterial using Immunohistochemical antibody staining.

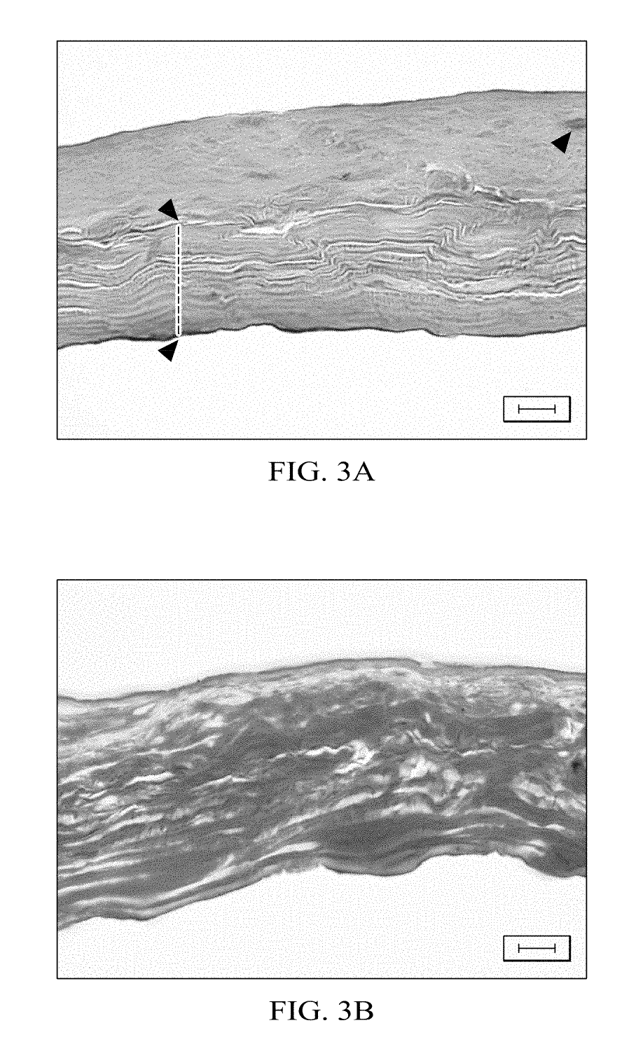

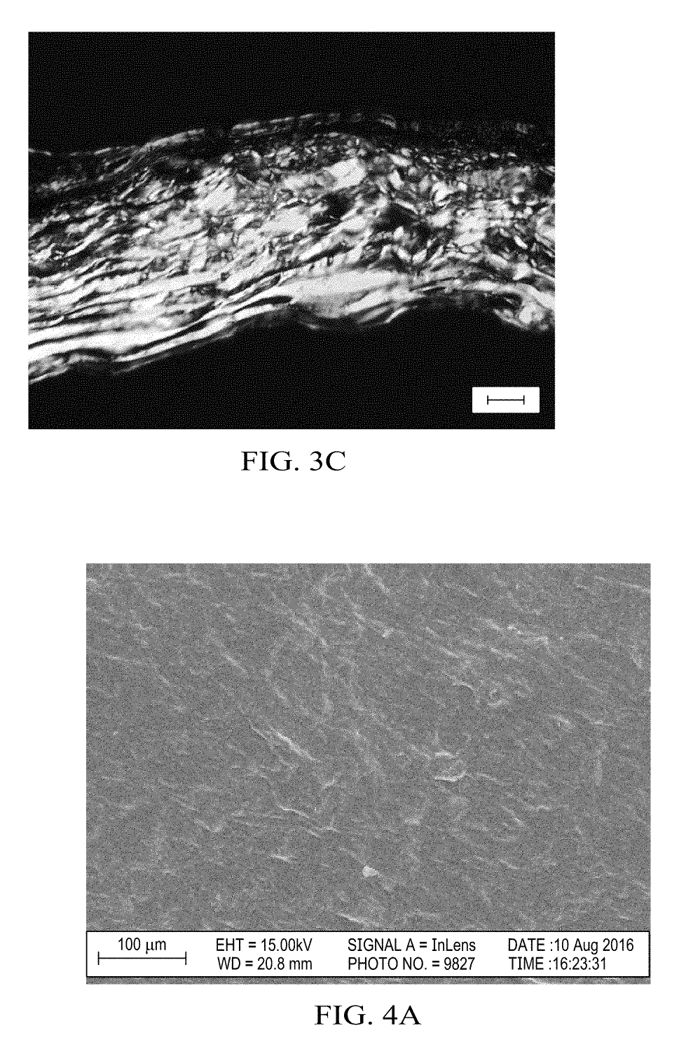

FIG. 3A and FIG. 3B show the bilateral histoarchitecture is evident as being retained and the absence of cell and cellular debris confirms the process renders the material acellular.

FIG. 3C shows Pico Sirius red birefrigence staining in H shows the relative ratio of collagen 111 to collagen 1 as would be expected in a Neotenic material.

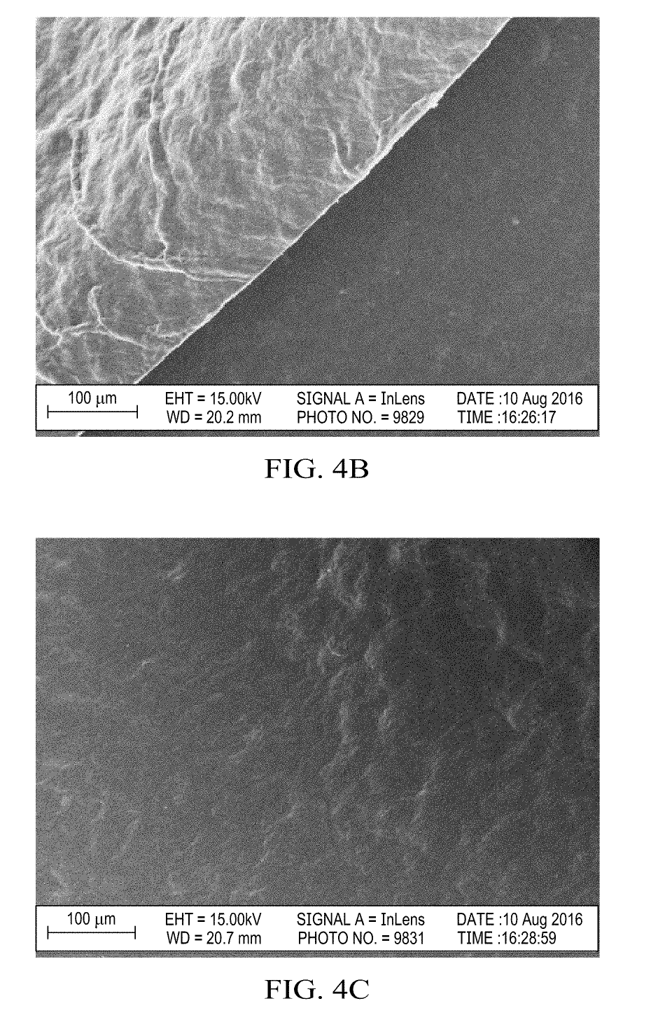

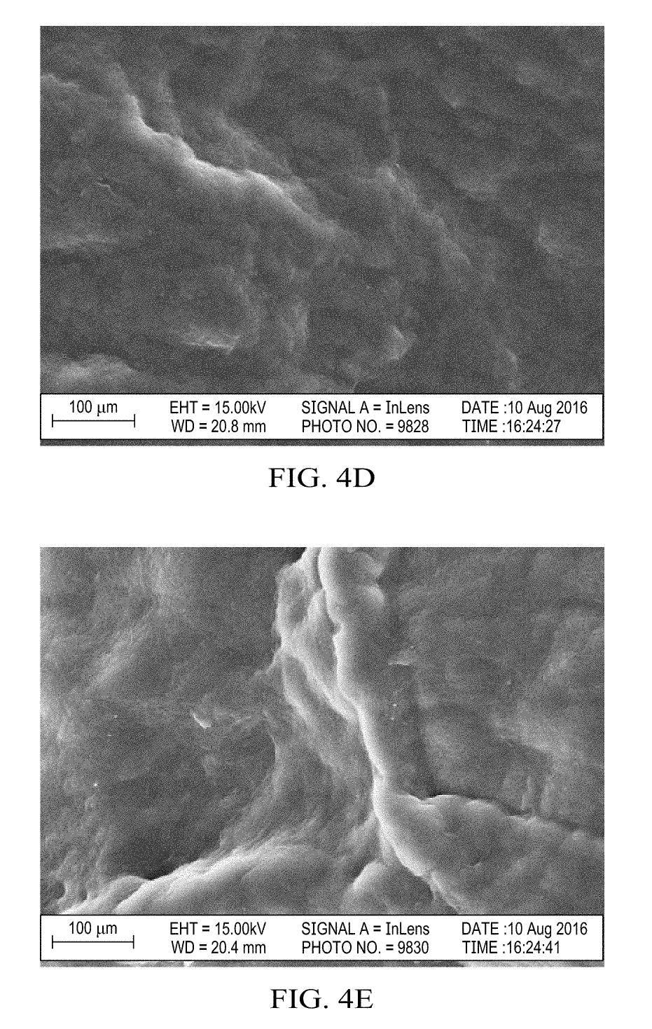

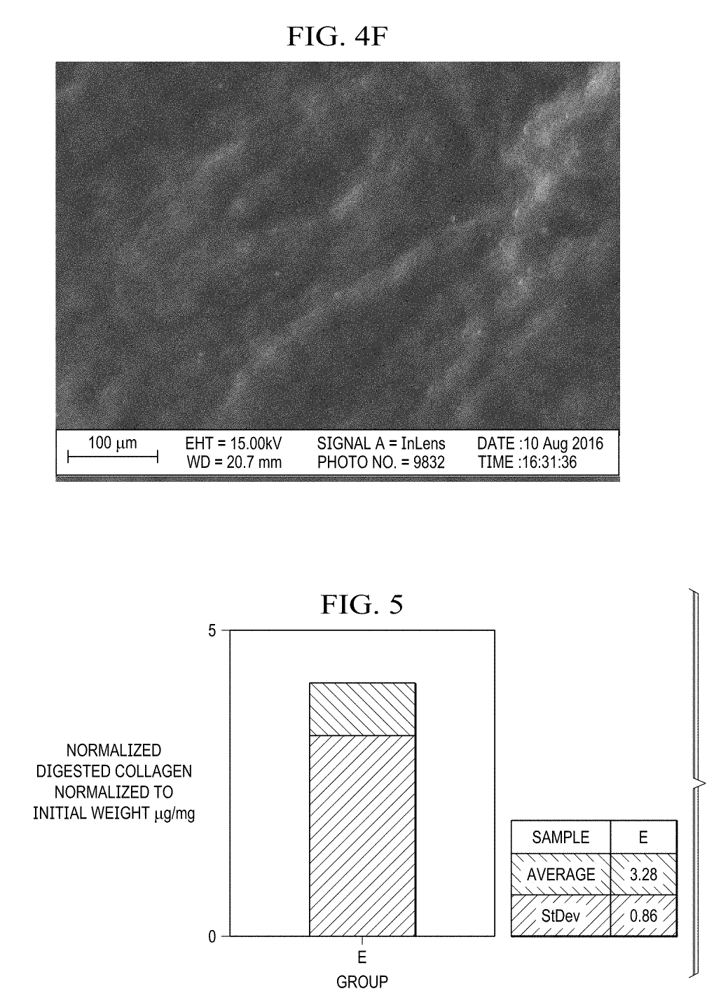

FIGS. 4A to 4F are SEM images, (E1, E2, E3), which show the decellurization of equine placental tissue.

FIG. 5 is a graph that shows the degradation of digested collagen normalized to initial weight, which demonstrates the enhanced collagen to normalized ratio.

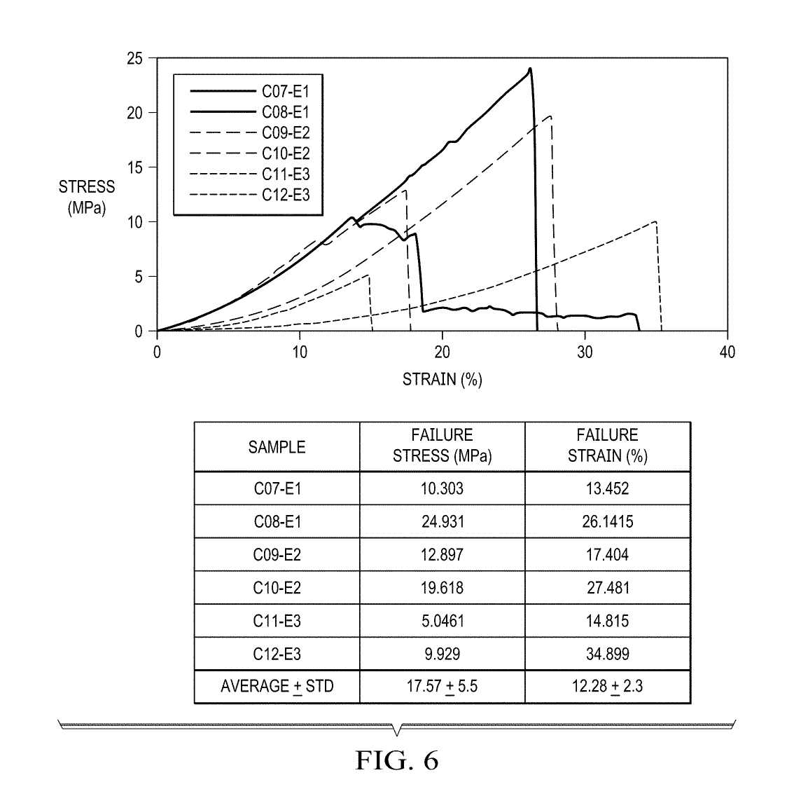

FIG. 6 is a graph that shows axial pull strength for the material of the present invention, which demonstrates the enhanced strength of the material.

A Cosmetic Product

Block-copolymers plus equine particulate 250-1000ic used after cosmetic procedure, such as dermal abrasion or chemical peel, etc. where Pluronic F127 (PF127) is prepared and physically blended with invention in volume to volume ration of 10-35%. Prepared missed compositions are either stored at room temperature above gelling temperature, allowing for alginate configuration. Prior to application of emollient topical application approx. every 4 hours alignate form or liquid form a thin layer of the mixed composition can be applied topically post procedurally thermo-gel (reverse phase) or spray, and allowed adequate time (less than 10 minutes) to dry after which the invention and active component will be distributed and proximal to the impacted area and have potential to beneficially impact consumer by minimized undesirable post procedural signs of irritation and enhance rate to which desirable effect of procedure are realized without impairing post procedural regimen of reapplication of emollient every 4 hours.

A Medical Research Product

Preparation of the invention in uniform diameter discs compatible with standard well plates of 6, 12 that are decellularized and prepared sterile are suitable and advantageous over readily available 2-D matrix substrate common products, like matrigel, as provided and has comparable composition and architecture, particularly known basement membrane components, including collagen IV, Collagen VII, Laminin And Fibronectin, but additionally retain 3-D architecture offering an improved biomimetic properties. Such a product is advantageous and offers chance to improve the value of preclinical toxicity studies for predicting clinical events. Additionally, improved cell culture is advantageous to ongoing medical research in the progression of stem cell phenotype transition during oncogenesis as well as regenerative medicine research focused on expansion of cell lines and culture of stem cells benefitting directly and indirectly regenerative medicine research. Multi-layer configurations, and side specific orientation variations which leverage exposure of various surface topology, porosity, pore kinetics, adhesion properties, adsorption properties, and ligand binding potential can be leveraged to aid understanding of the hierarchy related to structural-functional complexity and interdependence that is the basis for in vivo and in vitro simulation of cell-matrix interactions under specific conditions. Available 2-D soluble products omit to simulate key structural cues and precursors whereas insufficiently decellularized or alter prepared structural tissue material products often elicits initiation of DAMP cascade and/or inflammatory or fibrosis related cellular cascades. Only is a sufficient structurally and function preserved tissue material devoid of low molecular weight peptides and non-self-genetic material that is a reservoir of bound bioactive peptides and both bio-conductive and bio-inductive provide adequate conditions for simulation of mechanical transduction pathways, cell-to-cell or cell-to-matrix interaction, all which are necessary elements in accomplishing an in vitro biometric set up that has the potential to simulate events that are truly relevant and representative to in vivo events and interaction which are critical to new medical discovery and understanding that supports development of new medical solution and tools, as well as improving current methods of identifying sufficient safety profiles early in development before sufficient time and resources are allocated.

Osteogenic Device

Pluronic preparation blended with osteo-inductive agent such as DBM prepared via chemical demineralization or dry mill shearing of hydro-appetite and the biomaterial, which would provide substantial improvement over available flow able osteo-inductive products that are physically mixed, because of the additive and increased osteo-inductive potential contributed by the biomaterial and added benefit of the anti-microbial peptides and resultant bacteriostatic and bacteriosydal resultant product features that commercial available carrier-osteo-inductive agent physically blended compositions can potentially offer.

Pharmaceutical Composition

Solubility of the biomaterial and isolation of bioactive peptides having desirable individual or collective properties which can be used for additive function action to drug formulations, can be used to replace traditional formulations (antimicrobial peptides lack disadvantages of resistance and cellular immunity based mechanism of action present) are potentials for, or used to improve the pharmokinetis associated with active ingredient release profiles of oral and transdermal administered formulations by solubilizing the biomaterial in controlled fashion after uniform section of the material is performed and exposing it to 1M glacier acetic acid while agitating to sufficiently solubilize the material to its secondary structure by which preserving inherent self-assembly potential via fibrillogenesis. Temporary preservation of the solubilized fibular material is optimized by introduction of polar ionic solution which will inhibit formation of covalent bonds between polar organic molecules that otherwise would be difficult to control and inhibit. While in a controlled solubilized state a variety of additive can be introduced including not limited to addition active ingredient analgesic, antimicrobial compound, intact particulate of the starting invention material. Post loading of additive, physiological state, proper neutralization of the glacier acetic acid with 1M NAOH and salt precipitation during dehydration via air drying, solvent dehydration or lyophilization will sufficient restore physiological conditions necessary for fibro-genesis to occur and remove ions inhibiting covalent bond formation that occurs during fibrillogenesis. During and following fibrillogenesis events additive introduced will be tethered or bound via formation of covalent bonds in contrast to hydrogen bonding that support most carrier agent compositions. The benefit of the covalently bonded additive is multifaceted. It preserves active components that are low molecular weight and limited stability susceptible to being consumed post administration or placement during acute inflammatory cascades, it also reduces the potential to exacerbate or prolong inflammatory response compared to what configurations that have an immediate bolus release and availability of a bioactive component (bmp) and the subsequent compensation in product design by overdosing to compensate for loss during inflammation therefore reducing risks to patient of over dose and consequence of inflated costs of over designed product configuration and wastefulness of natural biological resource. The tethered or bound agent is also available naturally and has increased potential to persist and provide benefit at numerous points in the healing cascade therefore not just indirectly impacting the quality of host tissue by slowing down reformation and inflammation, but directly influencing the quality and rate of the formed tissue 14+ days after administration after inflammation has resolved. Long term the formation of quality host tissue for non-vascular limited functioning scar tissue is the essential to achieving long term success with surgical therapy vs. short term success currently achieved and necessity for subsequent continued intervention to address formation of post-surgical scar tissue which ultimately is excised surgically and results in negative feedback loop for patients who elect survival therapy intervention.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An acellular or decellularized biomaterial produced by the process that comprises:
obtaining placental tissue from an equine animal, which tissue sample comprises an extracellular matrix, and
decellularizing the tissue sample to retain structural and functional integrity while removing sufficient cellular components of the sample to be suitable for clinical use.

2. The decellularized biomaterial of claim 1, wherein the decellularizing comprises subjecting the placental tissue to an alkaline treatment.

3. The decellularized biomaterial of claim 1, wherein the process further comprises subjecting the sample to sterilization.

4. The decellularized biomaterial of claim 1, further comprising devitalized cells.

5. The decellularized biomaterial of claim 1, further comprising adding to the acellular or decellularized biomaterial at least one of: one or more block-copolymers, one or more osteogenic agent or one or more osteoinductive agents.

6. A tissue graft comprising extracellular matrix components derived from a placental tissue from an equine animal, wherein the graft has been processed to decellularize the tissue graft and retain structural and functional integrity.

7. An isolated, decellularized equine placental tissue extracellular membrane, wherein the membrane is inductive and conductive and at least one of: one or more block-copolymers, one or more osteogenic agent or one or more osteoinductive agents.

8. The isolated tissue of claim 6, wherein the extracellular matrix is alpha-Gal negative.

9. The isolated tissue of claim 6, wherein the extracellular matrix includes basement membrane.

10. The isolated tissue of claim 6, wherein the extracellular matrix is infused with, coated with, or attached to an agent xenogenic to equine placental tissue that is a growth factor, a cytokine, a chemokine, a protein, a carbohydrate, a sugar, a steroid, an antimicrobial agent, a synthetic polymer, an adhesive, a drug or a human agent.

11. The isolated tissue of claim 6, wherein the agent is a cell, optionally a human cell.

12. The isolated tissue of claim 6, wherein the extracellular membrane is a sheet.

13. The isolated tissue of claim 11, wherein the sheet includes perforations.

14. The isolated tissue of claim 6, wherein the extracellular membrane is a dry powder.

15. The isolated tissue of claim 6, wherein the extracellular membrane is a reconstituted gel.

16. The isolated tissue of any one of claim 6, wherein the extracellular membrane is sterile.

17. A package containing an isolated, sterile, decellularized equine placental tissue extracellular membrane, wherein the membrane is inductive and conductive.

18. The package of claim 17, wherein the isolated, sterile, decellularized equine placental tissue extracellular membrane is a sheet of isolated, decellularized Equine placental tissue equine tissue extracellular membrane.

19. The package of claim 17, wherein the isolated, sterile, decellularized Equine placental tissue equine tissue extracellular membrane is a dry powder.

20. The package of claim 17, wherein the isolated, sterile, decellularized equine placental tissue extracellular membrane is a gel.

21. A sterile medical implant comprising a sterile, isolated, decellularized equine placental tissue extracellular membrane, wherein the membrane is inductive and conductive.

22. The sterile medical implant of claim 19, wherein the implant is a biocompatible sheet, mesh, gel, graft, tissue or device.

23. A material coated with, impregnated with, encapsulating, or having attached thereto an isolated, sterile, decellularized equine placental tissue extracellular membrane, wherein the membrane is inductive and conductive.

24. A tissue culture system comprising: (a) an decellularized equine placental tissue extracellular membrane, (b) tissue culture medium, and (c) mammalian cells, wherein the membrane is inductive and conductive.

25. The tissue culture system of claim 22, wherein the mammalian cells are human cells.

26. A tissue culture medium conditioned with an isolated isolated, sterile, decellularized equine placental tissue extracellular membrane, wherein the membrane is inductive and conductive.

27. A device comprising at least two sheets of an isolated, sterile, decellularized equine placental tissue extracellular membrane laminated to one another, wherein the membrane is inductive and conductive.

28. A product prepared by isolating decellularized equine placental tissue extracellular membrane, and sterilizing the decellularized equine placental tissue extracellular membrane, wherein the membrane is inductive and conductive.

29. A method of preparing a biologic material comprising:
obtaining a tissue sample from an equine, which tissue sample comprises extracellular matrix;
decellularizing the sample forming a decellularized extracellular membrane to remove sufficient cellular components of the sample to reduce or eliminate antigenicity of the biomaterial as a xenograft, wherein the membrane is inductive and conductive; and
optionally adding to the decellularized extracellular membrane at least one of: one or more block-copolymers, one or more osteogenic agent, or one or more osteoinductive agents.

30. The method of claim 28, further comprising performing the decellularization in a manner to retain structural and functional integrity of the decellularized extracellular matrix membrane sufficient to permit the decellularized extracellular membrane to be useful as a matrix upon and within which cells can grow.

31. The method of claim 29, further comprising homogenizing the decellularized extracellular membrane to form a powder.

32. The method of claim 30, further comprising reconstituting the powder as a gel.

33. The method of claim 29, further comprising sterilizing the decellularized extracellular membrane.

34. The method of claim 29, further comprising attaching the decellularized extracellular membrane to an agent xenogenic to an equine.

35. The method of claim 28, wherein the extracellular membrane is α-Gal negative.

36. The method of claim 28, wherein the decellularlized equine extracellular membrane has a surface area of greater than 1,000 cm².

37. The method of claim 28, wherein the decellularlized equine extracellular membrane does not collapse during isolation.

38. The method of claim 28, wherein the decellularlized equine extracellular membrane is not used for ophthalmic use.

39. A method of treating a wound, burn, or surgical location comprising:
obtaining a decellularized equine placental tissue extracellular membrane to remove sufficient cellular components of the sample to reduce or eliminate antigenicity of the biomaterial as a xenograft, wherein the membrane is inductive and conductive;
placing the decellularized equine placental tissue extracellular membrane in, or, or about the wound, burn, or surgical location to treat the wound, burn, or surgical location; and
optionally adding to the decellularized extracellular membrane at least one of: one or more block-copolymers, one or more osteogenic agent, or one or more osteoinductive agents.

40. The method of claim 38, further comprising performing the decellularization in a manner to retain structural and functional integrity of the decellularized extracellular matrix membrane sufficient to permit the decellularized extracellular membrane to be useful as a matrix upon and within which cells can grow.

41. The method of claim 38, further comprising homogenizing the decellularized extracellular membrane to form a powder.

42. The method of claim 38, further comprising reconstituting the powder as a gel.

43. The method of claim 38, further comprising sterilizing the decellularized extracellular membrane.

44. The method of claim 38, further comprising attaching the decellularized extracellular membrane to an agent xenogenic to an equine.

45. The method of claim 38, wherein the decellularlized equine extracellular membrane is not used for ophthalmic use.
